(19) United States
(12) Patent Application Publication
(10) Pub. No.: US 2007/0004616 A1
(43) Pub. Date: Jan. 4, 2007

(54) GLP-1 pharmaceutical compositions

(76) Inventors: Roland Cherif-Cheikh, Castelldefels (ES); Jose Antonio Cordero Rigol, Rubi (Barcelona) (ES); Maria Dolores Tobalina Maestre, Barcelona (ES); Resurreccion Alloza Miravete, Amer (ES); Frederic Lacombe, Sant Cugat del Valles (ES)

Correspondence Address: Brian R. Morrill, Esq.;Biomeasure Incorporated, 27 Maple Street, Milford, MA, 01757-3650, US

(21) Appl. No.: 11/173,744

(22) Filed: Jun. 30, 2005

(51) Int. Cl. A61K 38/26

(52) U.S. Cl. 514/6; 514/12

(57) ABSTRACT

The present invention is directed to peptide analogues of glucagon-like peptide-1, the pharmaceutically-acceptable salts thereof, to methods of using such analogues to treat mammals and to pharmaceutical compositions useful therefor comprising said analogues.

### BACKGROUND OF THE INVENTION

The present invention is directed to peptide analogues of glucagon-like peptide-1, the pharmaceutically-acceptable salts thereof, to methods of using such analogues to treat mammals and to pharmaceutical compositions useful therefor comprising said analogues.

Glucagon-like peptide-1(7-36) amide (GLP-1) is synthesized in the intestinal L-cells by tissue-specific post-translational processing of the glucagon precursor preproglucagon (Varndell, J. M., et al., J. Histochem Cytochem, 1985:33:1080-6) and is released into the circulation in response to a meal. The plasma concentration of GLP-1 rises from a fasting level of approximately 15 pmol/L to a peak postprandial level of 40 pmol/L. It has been demonstrated that, for a given rise in plasma glucose concentration, the increase in plasma insulin is approximately threefold greater when glucose is administered orally compared with intravenously (Kreymann, B., et al., Lancet 1987:2, 1300-4). This alimentary enhancement of insulin release, known as the incretin effect, is primarily humoral and GLP-1 is thought to be the most potent physiological incretin in humans. In addition to the insulinotropic effect, GLP-1 suppresses glucagon secretion, delays gastric emptying (Wettergren A., et al., Dig Dis Sci 1993:38:665-73) and may enhance peripheral glucose disposal (D'Alessio, D. A. et al., J. Clin Invest 1994:93:2293-6).

In 1994, the therapeutic potential of GLP-1 was suggested following the observation that a single subcutaneous (s/c) dose of GLP-1 could completely normalize postprandial glucose levels in patients with non-insulin-dependent diabetes mellitus (NIDDM) (Gutniak, M. K., et al., Diabetes Care 1994:17:1039-44). This effect was thought to be mediated both by increased insulin release and by a reduction in glucagon secretion. Furthermore, an intravenous infusion of GLP-1 has been shown to delay postprandial gastric emptying in patients with NIDDM (Williams, B., et al., J. Clin Endo Metab 1996:81:327-32). Unlike sulphonylureas, the insulinotropic action of GLP-1 is dependent on plasma glucose concentration (Holz, G. G. 4^{th}, et al., Nature 1993:361:362-5). Thus, the loss of GLP-1-mediated insulin release at low plasma glucose concentration protects against severe hypoglycemia. This combination of actions gives GLP-1 unique potential therapeutic advantages over other agents currently used to treat NIDDM.

Numerous studies have shown that when given to healthy subjects, GLP-1 potently influences glycemic levels as well as insulin and glucagon concentrations (Orskov, C, Diabetologia 35:701-711,1992; Holst, J. J., et al., Potential of GLP-1 in diabetes management in Glucagon III, Handbook of Experimental Pharmacology, Lefevbre P J, Ed. Berlin, Springer Verlag, 1996, p. 311-326), effects which are glucose dependent (Kreymann, B., et al., Lancet ii: 1300-1304, 1987; Weir, G. C., et al., Diabetes 38:338-342, 1989). Moreover, it is also effective in patients with diabetes (Gutniak, M., N. Engl J Med 226:1316-1322, 1992; Nathan, D. M., et al., Diabetes Care 15:270-276, 1992), normalizing blood glucose levels in type 2 diabetic subjects (Nauck, M. A., et al., Diagbetologia 36:741-744, 1993), and improving glycemic control in type 1 patients (Creutzfeldt, W. O., et al., Diabetes Care 19:580-586,1996), demonstrating its ability to, inter alia, increase insulin sensitivity/reduce insulin resistance. GLP-1 and agonists thereof have been proposed for use in subjects at risk for developing non-insulin dependent diabetes (see WO 00/07617) as well as for the treatment of gestational diabetes mellitus (U.S. Patent Pub. No. 20040266670).

In addition to the foregoing, there are a number of therapeutic uses in mammals, e.g., humans, for which GLP-1 and agonists thereof have been suggested, including, without limitation: improving learning, enhancing neuro-protection, and/or alleviating a symptom of a disease or disorder of the central nervous system, e.g., through modulation of neurogenesis, and e.g., Parkinson's Disease, Alzheimer's Disease, Huntington's Disease, ALS, stroke, ADD, and neuropsychiatric syndromes (U.S. Patent Pub. No.'s 20050009742 and 20020115605); converting liver stem/progenitor cells into functional cells pancreatic (WO03/033697); preventing beta-cell deterioration (U.S. Patent Pub. No.'s 20040053819 and 20030220251) and stimulation of beta-cell proliferation (U.S. Patent Pub. No. 20030224983); treating obesity (U.S. Patent Pub. No. 20040018975; WO98/19698); suppressing appetite and inducing satiety (U.S. Patent Pub. No. 20030232754); treating irritable bowel syndrome (WO 99/64060); reducing the morbidity and/or mortality associated with myocardial infarction (US Patent Pub No. 20040162241, WO98/08531) and stroke (see WO 00/16797); treating acute coronary syndrome characterized by an absence of Q-wave myocardial infarction (U.S. Patent Pub. No. 20040002454); attenuating post-surgical catabolic changes (U.S. Pat. No. 6,006,753); treating hibernating myocardium or diabetic cardiomyopathy (U.S. Patent Pub. No. 20050096276); suppressing plasma blood levels of norepinepherine (U.S. Patent Pub. No.20050096276); increasing urinary sodium excretion, decreasing urinary potassium concentration (U.S. Patent Pub. No. 20050037958); treating conditions or disorders associated with toxic hypervolemia, e.g., renal failure, congestive heart failure, nephrotic syndrome, cirrhosis, pulmonary edema, and hypertension (U.S. Patent Pub. No. 20050037958); inducing an inotropic response and increasing cardiac contractility (U.S. Patent Pub. No. 20050037958); treating polycystic ovary syndrome (U.S. Patent Pub. No.'s 20040266678 & 20040029784); treating respiratory distress (U.S. Patent Pub. No. 20040235726); improving nutrition via a non-alimentary route, i.e., via intravenous, subcutaneous, intramuscular, peritoneal, or other injection or infusion (U.S. Patent Pub. No.20040209814); treating nephropathy (U.S. Patent Pub. No. 20040209803); treating left ventricular systolic dysfunction, e.g., with abnormal left ventricular ejection fraction (U.S. Patent Pub. No. 20040097411); inhibiting antro-duodenal motility, e.g., for the treatment or prevention of gastrointestinal disorders such as diarrhea, postoperative dumping syndrome and irritable bowel syndrome, and as premedication in endoscopic procedures (U.S. Patent Pub. No. 20030216292); treating critical illness polyneuropathy (CIPN) and systemic inflammatory response syndrome (SIRS) (U.S. Patent Pub. No. 20030199445); modulating triglyceride levels and treating dyslipidemia (U.S. Patent Pub. No.'s 20030036504 and 20030143183); treating organ tissue injury caused by reperfusion of blood flow following ischemia (U.S. Patent Pub. No. 20020147131); treating coronary heart disease risk factor (CHDRF) syndrome (U.S. Patent Pub. No. 20020045636); and others. GLP-1 is, however, metabolically unstable, having a plasma half-life (t_{1/2}) of only 1-2 min in vivo. Exogenously administered GLP-1 is also rapidly degraded (Deacon, C. F., et al., Diabetes 44:1126-1131, 1995). This metabolic instability limits the therapeutic potential of native GLP-1.

A number of attempts have been taken to improve the therapeutic potential of GLP-1 and its analogs through improvements in formulation. For example, International patent publication no. WO 01/57084 describes a process for producing crystals of GLP-1 analogues which are said to be useful in the preparation of pharmaceutical compositions, such as injectable drugs, comprising the crystals and a pharmaceutical acceptable carrier. Heterogeneous micro crystalline clusters of GLP-1(7-37)OH have been grown from saline solutions and examined after crystal soaking treatment with zinc and/or m-cresol (Kim and Haren, Pharma. Res. Vol. 12 No. 11 (1995)). Crude crystalline suspensions of GLP(7-36)NH₂ containing needle-like crystals and amorphous precipitation have been prepared from phosphate solutions containing zinc or protamine (Pridal, et. al., International Journal of Pharmaceutics Vol. 136, pp. 53-59 (1996)). European patent publication no. EP 0619322A2 describes the preparation of micro-crystalline forms of GLP-1(7-37)OH by mixing solutions of the protein in pH 7-8.5 buffer with certain combinations of salts and low molecular weight polyethylene glycols (PEG). U.S. Pat. No. 6,566,490 describes seeding microcrystals of, inter alia, GLP-1 which are said to aid in the production of purified peptide products. U.S. Pat. No. 6,555,521 (U.S. '521) discloses GLP-1 crystals having a tetragonal flat rod or a plate-like shape which are said to have improved purity and to exhibit extended in vivo activity. U.S. '521 teaches that such crystals are relatively uniform and remain in suspension for a longer period of time than prior crystalline clusters and amorphous crystalline suspensions which were said to settle rapidly, aggregate or clump together, clog syringe needles and generally exacerbate unpredictable dosing.

A biodegradable triblock copolymer of poly [(dl-lactide-co-glycolide)-β-ethylene glycol-β-(-lactide-co-glycolide)] has been suggested for use in a controlled release formulation of GLP-1. However like other polymeric systems, the manufacture of triblock copolymer involves complex protocols and inconsistent particulate formation.

Similarly, biodegradable polymers, e.g., poly(lactic-co-glycolic acid) (PLGA), have also been suggested for use in sustained delivery formulations of peptides. However the use of such biodegradable polymers has been disfavored in the art since these polymers generally have poor solubility in water and require water-immiscible organic solvents, e.g., methylene chloride, and/or harsh preparation conditions during manufacture. Such organic solvents and/or harsh preparation conditions are considered to increase the risk of inducing conformational change of the peptide or protein of interest, resulting in decreased structural integrity and compromised biological activity. (Choi et al., Pharm. Research, Vol. 21, No. 5, (2004).) Poloxamers have been likewise faulted. (Id.)

The GLP-1 compositions described in the foregoing references are less than ideal for preparing pharmaceutical formulations of GLP's since they tend to trap impurities and/or are otherwise difficult to reproducibly manufacture and administer. Also, GLP analogs are known to induce nausea at elevated concentrations, thus there is a need to provide a sustained drug effect with reduced initial plasma concentrations. Hence, there is a need for GLP-1 formulations which are more easily and reliably manufactured, that are more easily and reproducibly administered to a patient, and that provide for reduced initial plasma concentrations in order to reduce or eliminate unwanted side-effects.

### SUMMARY OF THE INVENTION

The invention may be summarized in the following paragraphs as well as the claims. Accordingly, it is a first object of the invention to provide a pharmaceutical composition comprising a GLP-1 analog according to formula (I):
(Aib^{8,35})hGLP-1(7-36)NH₂ (I)
or a pharmaceutically acceptable salt thereof, wherein the formulation of said composition provides for superior manufacturing, administration, pharmacokinetic and pharmacodynamic properties, as well as attenuated negative side-effects.

In a first aspect of said first object the invention provides for a pharmaceutical composition having an improved drug release profile, preferably with a reduced initial burst.

In a second aspect of said first object the invention provides for pharmaceutical composition comprising a compound of formula (I) having an extended duration of action.

In a third aspect of said first object the invention provides for a pharmaceutical composition comprising a compound of formula (I) or a pharmaceutically acceptable salt thereof and a pharmaceutically acceptable carrier or diluent. Preferably said carrier or diluent comprises water.

In a first preferred embodiment of said third aspect of said first object said pharmaceutical composition further comprises zinc. More preferably, said pharmaceutical composition comprises an aqueous mixture, suspension or solution, wherein said compound of formula (I) is present at a concentration of approximately 0.5%-30% (w/w). More preferably the concentration of said compound of formula (I) in said aqueous mixture, suspension or solution is approximately 1%, 2%, 3%, 4%, 5%, 6%, 7%, 8%, 9%,10%, 11%, 12%, 13%,14%, 15%,16%, 17%, 18%, 19%, 20%, 21%, 22%, 23%, 24%, 25%, 26%, 27%, 28%, 29%, or 30% (w/w). More preferably, the concentration of said compound of formula (I) in said aqueous solution is approximately 1%, 2%, 3%, 4%, 5%, 6%, 7%, 8%, 9%,10%, 11%, 14%, 15%, 16%, 19%, 20%, 21%, 22%, 23%, 24%, 25%, 26%, 29%, or 30% (w/w). More preferably still, the concentration of said compound of formula (I) in said aqueous solution is approximately 1%, 2%, 3%, 4%, 5%, 6%, 9%, 10%, 11%, 22%, 23%, 24%, 25%, or 26% (w/w). Even more preferably still, the concentration of said compound of formula (I) in said aqueous solution is approximately 1%, 2%, 3%, 4%, 5%, 6%, 10%, 22%, 23%, 24%, 25%, or 26% (w/w). Still more preferably, the concentration of said compound of formula (I) in said aqueous solution is approximately 1%, 2%, 5%, 10%, 23% or 25% (w/w).

In a second preferred embodiment of said third aspect of said first object, said pharmaceutical composition further comprises zinc, wherein the molar ratio of said compound of formula (I) to zinc in said pharmaceutical composition ranges from approximately 6:1 to approximately 1:1. More preferably, said ratio ranges from approximately 5.5:1 to approximately 1:1. More preferably still, said ratio ranges from approximately 5.4:1 to approximately 1.5:1. Even more preferably still, said ratio is approximately 5.4:1, 4.0:1, or 1.5:1. Most preferably, said ratio is approximately 1.5:1.

Preferably, in said second preferred embodiment of said third aspect of said first object, said zinc is provided as zinc chloride or zinc acetate. More preferably, said zinc acetate is provided as ZnAc₂.2 H₂O.

Preferably, in both of said first and second preferred embodiments of said third aspect of said first object, the pH of said pharmaceutical composition is adjusted upward using a base. More preferably, said pH adjustment is made using NaOH. More preferably still, the pH of said pharmaceutical composition is adjusted with NaOH such that, when diluted to approximately ½ initial concentration using 0.9% NaCl, a pH value of approximately 5.0-5.5 is obtained using direct potentiometric determination.

In a first preferred embodiment of said second aspect of said first object, the invention features a pharmaceutical composition according to said third aspect, including, independently for each occurrence, each of said preferred embodiments of said third aspect, wherein the composition is formulated such that the compound according to formula (I) is released within a subject in need thereof, e.g., a mammal, preferably a human, for an extended period of time. Preferably said release of said compound extends for at least one hour, more preferably at least 4, 6, 12, or 24 hours. More preferably still, said composition is formulated such that the compound according to formula (I) is released within a subject for at least 36, 48, 60, 72, 84, or 96 hours. More preferably still, said composition is formulated such that the compound according to formula (I) is released within a subject for at least approximately 5, 6, 7, 8, 9, 10, 11, 12, 13, or 14 days. More preferably still, said composition is formulated such that the compound according to formula (I) is released within a subject for at least approximately 2, 3 or 4 weeks.

It is a second object of the present invention to provide for a method of eliciting a GLP-1 agonist effect, said method comprising contacting a receptor of the GLP-1 (7-36)NH₂ ligand with the compound according to formula (I), said compound according to formula (I) being provided to said receptor, directly or indirectly, via a composition according to said third aspect, including, independently for each occurrence, each of said preferred embodiments of said third aspect.

In a first preferred embodiment of said second object of the invention, said receptor of the GLP-1(7-36)NH₂ ligand is present in an animal subject, preferably a primate, more preferably a human being. Thus, in this embodiment the present invention provides a method of eliciting an agonist effect from a GLP-1 receptor in a subject in need thereof which comprises administering to said subject a composition of the instant invention, wherein said composition comprises an effective amount of a compound of formula (I) or a pharmaceutically acceptable salt thereof.

In a more preferred embodiment of said second object of the invention, said subject is a human afflicted with, or at risk of developing, a disease or condition selected from the group consisting of Type I diabetes, Type II diabetes, gestational diabetes, obesity, excessive appetite, insufficient satiety, and metabolic disorder. Preferably said disease is Type I diabetes or Type II diabetes.

In another more preferred embodiment of said second object of the invention, said subject is a human afflicted with, or at risk of developing, a disease selected from the group consisting of Type I diabetes, Type II diabetes, obesity, glucagonomas, secretory disorders of the airway, arthritis, osteoporosis, central nervous system disease, restenosis, neurodegenerative disease, renal failure, congestive heart failure, nephrotic syndrome, cirrhosis, pulmonary edema, hypertension, and disorders wherein the reduction of food intake is desired, a disease or disorder of the central nervous system, (e.g., through modulation of neurogenesis, and e.g., Parkinson's Disease, Alzheimer's Disease, Huntington's Disease, ALS, stroke, ADD, and neuropsychiatric syndromes), irritable bowel syndrome, myocardial infarction (e.g., reducing the morbidity and/or mortality associated therewith), stroke, acute coronary syndrome (e.g., characterized by an absence of Q-wave myocardial infarction, post-surgical catabolic changes, hibernating myocardium or diabetic cardiomyopathy, insufficient urinary sodium excretion, excessive urinary potassium concentration, conditions or disorders associated with toxic hypervolemia, (e.g., renal failure, congestive heart failure, nephrotic syndrome, cirrhosis, pulmonary edema, and hypertension), polycystic ovary syndrome, respiratory distress, nephropathy, left ventricular systolic dysfunction, (e.g., with abnormal left ventricular ejection fraction), gastrointestinal disorders such as diarrhea, postoperative dumping syndrome and irritable bowel syndrome, (i.e., via inhibition of antro-duodenal motility), critical illness polyneuropathy (CIPN), systemic inflammatory response syndrome (SIRS), dyslipidemia, organ tissue injury caused by reperfusion of blood flow following ischemia, and coronary heart disease risk factor (CHDRF) syndrome.

In another aspect of said second object, the invention features a method of converting liver stem/progenitor cells into functional pancreatic cells, of preventing beta-cell deterioration and of stimulating beta-cell proliferation, of suppressing plasma blood levels of norepinepherine, of inducing an inotropic response and of increasing cardiac contractility, of improving nutrition via a non-alimentary route, (e.g., via intravenous, subcutaneous, intramuscular, peritoneal, or other injection or infusion rout), of pre-treating a subject to undergo an endoscopic procedures, and of modulating triglyceride levels, in a subject in need thereof, said method comprising administering to said subject a formulation of the present invention comprising an effective amount of a compound of formula (I) or a pharmaceutically acceptable salt thereof. Preferably said subject is a mammalian animal, more preferably a primate, more preferably still a human being.

With the exception of the N-terminal amino acid, all abbreviations (e.g. Ala) of amino acids in this disclosure stand for the structure of —NH—CH(R)—CO—, wherein R is the side chain of an amino acid (e.g., CH₃ for Ala). For the N-terminal amino acid, the abbreviation stands for the structure of (R²R³)—N—CH(R)—CO—, wherein R is a side chain of an amino acid and R² and R³ are as defined above, except when A⁷ is Ura, Paa or Pta, in which case R² and R³ are not present since Ura, Paa and Pta are considered here as des-amino amino acids. Amp, 1Nal, 2Nal, Nle, Cha, 3-Pal, 4-Pal and Aib are the abbreviations of the following a-amino acids: 4-amino-phenylalanine, β-(1-naphthyl)alanine, β-(2-naphthyl)alanine, norleucine, cyclohexylalanine, β-(3-pyridinyl)alanine, β-(4-pyridinyl)alanine and α-aminoisobutyric acid, respectively. Other amino acid definitions are: Ura is urocanic acid; Pta is (4-pyridylthio) acetic acid; Paa is trans-3-(3-pyridyl) acrylic acid; Tma-His is N,N-tetramethylamidino-histidine; N-Me-Ala is N-methyl-alanine; N-Me-Gly is N-methyl-glycine; N-Me-Glu is N-methyl-glutamic acid; Tle is tert-butylglycine; Abu is α-aminobutyric acid; Tba is tert-butylalanine; Orn is ornithine; Aib is α-aminoisobutyric acid; β-Ala is β-alanine; Gaba is γ-aminobutyric acid; Ava is 5-aminovaleric acid; Ado is 12-aminododecanoic acid, Aic is 2-aminoindane-2-carboxylic acid; Aun is 11-aminoundecanoic acid; and Aec is 4-(2-aminoethyl)-1-carboxymethyl-piperazine, represented by the structure:

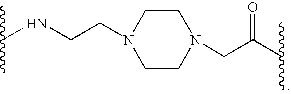

What is meant by Acc is an amino acid selected from the group of 1-amino-1-cyclopropanecarboxylic acid (A3c); 1-amino-1-cyclobutanecarboxylic acid (A4c); 1-amino-1-cyclopentanecarboxylic acid (A5c); 1-amino-1-cyclohexanecarboxylic acid (A6c); 1-amino-1-cycloheptanecarboxylic acid (A7c); 1-amino-1-cyclooctanecarboxylic acid (A8c); and 1-amino-1-cyclononanecarboxylic acid (A9c). In the above formula, hydroxyalkyl, hydroxyphenylalkyl, and hydroxynaphthylalkyl may contain 1-4 hydroxy substituents. COX⁵ stands for —C═OX⁵. Examples of —C═OX⁵ include, but are not limited to, acetyl and phenylpropionyl.

The full names for other abbreviations used herein are as follows: Boc for t-butyloxycarbonyl, HF for hydrogen fluoride, Fm for formyl, Xan for xanthyl, Bzl for benzyl, Tos for tosyl, DNP for 2,4-dinitrophenyl, DMF for dimethylformamide, DCM for dichloromethane, HBTU for 2-(1H-Benzotriazol-1-yl)-1,1,3,3-tetramethyl uronium hexafluorophosphate, DIEA for diisopropylethylamine, HOAc for acetic acid, TFA for trifluoroacetic acid, 2CIZ for 2-chlorobenzyloxycarbonyl, 2BrZ for 2-bromobenzyloxycarbonyl, OcHex for O-cyclohexyl, Fmoc for 9-fluorenylmethoxycarbonyl, HOBt for N-hydroxybenzotriazole; PAM resin for 4-hydroxymethylphenylacetamidomethyl resin; Tris for Tris(hydroxymethyl)aminomethane; and Bis-Tris for Bis(2-hydroxyethyl)amino-tris(hydroxymethyl)methane (i.e., 2-Bis(2-hydroxyethyl)amino-2-(hydroxymethyl)-1,3-propanediol).

The term “halo” or “halogen” encompasses fluoro, chloro, bromo and iodo.

The terms “(C₁-C₁₂)hydrocarbon moiety”, “(C₁-C₃₀)hydrocarbon moiety” and the like encompass branched and straight chain alkyl, alkenyl and alkynyl groups having the indicated number of carbons, provided that in the case of alkenyl and alkynyl there is a minimum of two carbons.

A peptide of this invention is also denoted herein by another format, e.g., (Aib^{8,35})hGLP-1(7-36)NH₂, with the substituted amino acids from the natural sequence placed between the first set of parentheses (e.g., Aib^{8,35} denotes that Aib is substituted for Ala⁸ and Gly³⁵ in hGLP-1). The abbreviation GLP-1 means glucagon-like peptide-1; hGLP-1 means human glucagon-like peptide-1. The numbers between the second set of parentheses refer to the number of amino acids present in the peptide (e.g., hGLP-1(7-36) refers to amino acids 7 through 36 of the peptide sequence for human GLP-1). The sequence for hGLP-1(7-37) is listed in Mojsov, S., Int. J. Peptide Protein Res,. 40, 1992, pp. 333-342. The designation “NH₂” in hGLP-1(7-36)NH₂ indicates that the C-terminus of the peptide is amidated. hGLP-1(7-36) means that the C-terminus is the free acid. In hGLP-1(7-38), residues in positions 37 and 38 are Gly and Arg, respectively, unless otherwise indicated.

### BRIEF DESCRIPTION OF THE DRAWING

FIG. 1 is a schematic illustration which depicts a syringe-based device useful for assuring homogeneity of semi-solid compositions of the invention.

### DETAILED DESCRIPTION

Synthesis of Peptides

Peptides useful for practicing the present invention can be and were prepared by standard solid phase peptide synthesis. See, e.g., Stewart, J. M., et al., Solid Phase Synthesis (Pierce Chemical Co., 2d ed. 1984). The substituents R² and R³ of the above generic formula may be attached to the free amine of the N-terminal amino acid by standard methods known in the art. For example, alkyl groups, e.g., (C₁-C₃₀)alkyl, may be attached using reductive alkylation. Hydroxyalkyl groups, e.g., (C₁-C₃₀)hydroxyalkyl, may also be attached using reductive alkylation wherein the free hydroxy group is protected with a t-butyl ester. Acyl groups, e.g., COE¹, may be attached by coupling the free acid, e.g., E¹COOH, to the free amine of the N-terminal amino acid by mixing the completed resin with 3 molar equivalents of both the free acid and diisopropylcarbodiimide in methylene chloride for one hour. If the free acid contains a free hydroxy group, e.g., p-hydroxyphenylpropionic acid, then the coupling should be performed with an additional 3 molar equivalents of HOBT.

When R¹ is NH—X²—CH₂—CONH₂, (i.e., Z⁰=CONH₂), the synthesis of the peptide starts with BocHN—X²—CH₂—COOH which is coupled to the MBHA resin. If R¹ is NH—X²—CH₂—COOH, (i.e., Z⁰=COOH) the synthesis of the peptide starts with Boc-HN—X²—CH₂—COOH which is coupled to PAM resin. For this particular step, 4 molar equivalents of Boc-HN—X²—COOH, HBTU and HOBt and 10 molar equivalents of DIEA are used. The coupling time is about 8 hours.

The protected amino acid 1-(N-tert-butoxycarbonyl-amino)-1-cyclohexane-carboxylic acid (Boc-A6c-OH) was synthesized as follows. 19.1 g (0.133 mol) of 1-amino-1-cyclohexanecarboxylic acid (Acros Organics, Fisher Scientific, Pittsburgh, Pa.) was dissolved in 200 ml of dioxane and 100 ml of water. To it was added 67 ml of 2N NaOH. The solution was cooled in an ice-water bath. 32.0 g (0.147 mol) of di-tert-butyl-dicarbonate was added to this solution. The reaction mixture was stirred overnight at room temperature. Dioxane was then removed under reduced pressure. 200 ml of ethyl acetate was added to the remaining aqueous solution. The mixture was cooled in an ice-water bath. The pH of the aqueous layer was adjusted to about 3 by adding 4N HCl. The organic layer was separated. The aqueous layer was extracted with ethyl acetate (1×100 ml). The two organic layers were combined and washed with water (2×150 ml), dried over anhydrous MgSO₄, filtered, and concentrated to dryness under reduced pressure. The residue was recrystallized in ethyl acetate/hexanes. 9.2 g of the pure product was obtained. 29% yield.

Boc-A5c-OH was synthesized in an analogous manner to that of Boc-A6c-OH. Other protected Acc amino acids can be prepared in an analogous manner by a person of ordinary skill in the art as enabled by the teachings herein.

In the synthesis of a peptide containing A5c, A6c and/or Aib, the coupling time is 2 hrs. for these residues and the residue immediately following them.

The substituents R² and R³ of the above generic formula can be attached to the free amine of the N-terminal amino acid by standard methods known in the art. For example, alkyl groups, e.g., (C₁-C₃₀)alkyl, can be attached using reductive alkylation. Hydroxyalkyl groups, e.g., (C₁-C₃₀)hydroxyalkyl, can also be attached using reductive alkylation wherein the free hydroxy group is protected with a t-butyl ester. Acyl groups, e.g., COX¹, can be attached by coupling the free acid, e.g., X¹COOH, to the free amine of the N-terminal amino acid by mixing the completed resin with 3 molar equivalents of both the free acid and diisopropylcarbodiimide in methylene chloride for about one hour. If the free acid contains a free hydroxy group, e.g., p-hydroxyphenylpropionic acid, then the coupling should be performed with an additional 3 molar equivalents of HOBT.

The following examples describe synthetic methods that can be and were used for making peptides with which the instant invention may advantageously be practiced, which synthetic methods are well-known to those skilled in the art. Other methods are also known to those skilled in the art. The examples are provided for the purpose of illustration and are not meant to limit the scope of the present invention in any manner.

Boc-βAla-OH, Boc-D-Arg(Tos)-OH and Boc-D-Asp(OcHex) were purchased from Nova Biochem, San Diego, Calif. Boc-Aun-OH was purchased from Bachem, King of Prussia, PA. Boc-Ava-OH and Boc-Ado-OH were purchased from Chem-Impex International, Wood Dale, Ill. Boc-2Nal-OH was purchased from Synthetech, Inc. Albany, Oreg.

### EXAMPLE 1

(Aib^{8,35})hGLP-1(7-36)NH₂

A detailed synthesis procedure for (Aib^{8,35})hGLP-1(7-36)NH₂ has been provided in International Patent Publication No. WO 00/34331 (PCT/EP99/09660), the contents of which are incorporated herein in their entirety. Briefly, the compound was synthesized on an Applied Biosystems (Foster City, Calif.) model 430A peptide synthesizer which was modified to do accelerated Boc-chemistry solid phase peptide synthesis. See Schnolzer, et al., Int. J. Peptide Protein Res., 90:180 (1992). 4-methylbenzhydrylamine (MBHA) resin (Peninsula, Belmont, Calif.) with the substitution of 0.91 mmol/g was used. The Boc amino acids (Bachem, Calif., Torrance, Calif.; Nova Biochem., LaJolla, Calif.) were used with the following side chain protection: Boc-Ala-OH, Boc-Arg(Tos)-OH, Boc-Asp(OcHex)-OH, Boc-Tyr(2BrZ)-OH, Boc-His(DNP)-OH, Boc-Val-OH, Boc-Leu-OH, Boc-Gly-OH, Boc-Gln-OH, Boc-Ile-OH, Boc-Lys(2CIZ)-OH, Boc-Thr(Bzl)-OH, Boc-Ser(Bzl)-OH, Boc-Phe-OH, Boc-Aib-OH, Boc-Glu(OcHex)-OH and Boc-Trp(Fm)-OH. The Boc groups were removed by treatment with 100% TFA for 2×1 min. Boc amino acids (2.5 mmol) were pre-activated with HBTU (2.0 mmol) and DIEA (1.0 mL) in 4 mL of DMF and were coupled without prior neutralization of the peptide-resin TFA salt. Coupling times were 5 min. except for the Boc-Aib-OH residues and the following residues, Boc-Lys(2CIZ)-OH and Boc-His(DNP)-OH wherein the coupling times were 2 hours.

At the end of the assembly of the peptide chain, the resin was treated with a solution of 20% mercaptoethanol/10% DIEA in DMF for 2×30 min. to remove the DNP group on the His side chain. The N-terminal Boc group was then removed by treatment with 100% TFA for 2×2 min. After neutralization of the peptide-resin with 10% DIEA in DMF (1×1 min), the formyl group on the side chain of Trp was removed by treatment with a solution of 15% ethanolamine/15% water/70% DMF for 2×30 min. The peptide-resin was washed with DMF and DCM and dried under reduced pressure. The final cleavage was done by stirring the peptide-resin in 10 mL of HF containing 1 mL of anisole and dithiothreitol (24 mg) at 0° C. for 75 min. HF was removed by a flow of nitrogen. The residue was washed with ether (6×10 mL) and extracted with 4N HOAc (6×10 mL).

The peptide mixture in the aqueous extract was purified on reverse-phase preparative high pressure liquid chromatography (HPLC) using a reverse phase VYDAC® C₁₈ column (Nest Group, Southborough, Mass.). The column was eluted with a linear gradient (20% to 50% of solution B over 105 min.) at a flow rate of 10 mL/min (Solution A=water containing 0.1% TFA; Solution B=acetonitrile containing 0.1% of TFA). Fractions were collected and checked on analytical HPLC. Those containing pure product were combined and lyophilized to dryness. In one example of synthesis of this compound, 135 mg of a white solid was obtained. Purity was 98.6% based on analytical HPLC analysis. Electro-spray mass spectrometer (MS(ES))S analysis gave the molecular weight at 3339.7 (in agreement with the calculated molecular weight of 3339.7).

Formulation Procedures—Part I

1. Materials

ZnCl₂, NaOH pellets, and hydrochloric acid, 35%, were obtained from Panreac Quimica, Barcelona, Spain. WFI (sterile water for injection/irrigation) was obtained from B. Braun Medical, Barcelona, Spain.

2. Preparation of Stock Solutions

A. Preparation of a ZnCl₂ Stock Solution with Concentration Between 1-4 mg/mL, Dissolving with HCl pH=3.

1. Add HCl 35% to WFI while stirring, to achieve pH=3. Measure pH to confirm.

2. In a volumetric flask, transfer a weighed amount of ZnCl2. Add enough HCl pH=3 (from step A.1) to volume and stir. Final concentration to be approximately 1-4 mg ZnCl2/mL. B. Preparation of a NaOH stock solution with concentration between 0.1-10 mg/mL, dissolving with water for injection.

1. In a volumetric flask, transfer a weighed amount of NaOH. Add enough WFI to volume and stir. Final concentration to be 0.1-10 mg NaOH/mL.

Preparation of compositions with 1-2-10% peptide and ZnCl2, without adjusting pH upward with base (e.g., from a freeze-dried vial)—Preparation of a freeze-dried composition with (Aib^{8,35})HGLP-1 (7-36)NH₂

C. 20 mg (Aib^{8,35})HGLP-1(7-36)NH₂/vial:

1. In a volumetric flask, transfer an exact volume of acetic acid. Add enough WFI to volume and stir. Final concentration to be 0.04% (VN).
2. In a volumetric flask, transfer a weighed amount of (Aib^{8,35})HGLP-1(7-36)NH₂ (acetate salt). Add sufficient 0.04% acetic acid (from step C.1), with stirring, to bring final concentration to 20 mg (Aib^{8,35})HGLP-1(7-36)NH₂/mL. After sterile filtration, 1 ml aliquots of the solution are transferred to lyophilization vials and freeze dried.
   D. 50 mg (Aib^{8,35})HGLP-1(7-36)NH₂/vial:
1. In a volumetric flask, transfer an exact volume of acetic acid. Add enough WFI to volume and stir. Final concentration to be 0.1% (VN)
2. In a volumetric flask, transfer a weighed amount of (Aib^{8,35})HGLP-1(7-36)NH₂. Add enough acetic acid 0.1% (from step D.1) to achieve a final concentration of 50 mg (Aib^{8,35})HGLP-1(7-36)NH₂/mL. After sterile filtration, 1 ml aliquots of the solution are transferred to lyophilization vials and freeze dried.
   Preparation of a ZnCl2 solution with a desired concentration:
1. In a volumetric flask, transfer an aliquot ZnCl2 stock solution. Sufficient HCl (pH=3, from step
   A.1) is added to achieve target composition in view of peptide raw material. (See F.1., below.)
2. A known weight of peptide is stirred with the necessary volume (100% of total volume of excipient) of solution of ZnCl2 from 1., to achieve the target concentration (e.g., 1%, 2% or 10% (w/w)).
   1% compositions: Take a vial with 20 mg (Aib^{8,35})HGLP-1 (7-36)NH₂ (step C) and add 2 mL of ZnCl2 solution of proper concentration (See F.1., below)
   2% compositions: Take a vial with 20 mg (Aib^{8,35})HGLP-1(7-36)NH₂ (step C) and add 1 mL of ZnCl2 solution of proper concentration (See F.1., below)
   10% compositions: Take a vial with 50 mg (Aib^{8,35})HGLP-1(7-36)NH₂ (step D) and add 0.45 mL of ZnCl2 solution of proper concentration (See F.1., below)
      Shake Until Dissolution.
      Compositions with 1-2-10% peptide and ZnCl2, without upward adjustment of pH (standard liquid composition method. See F.2., below)
      Preparation of a ZnCl2 solution with the appropriate concentration: Take an aliquot of the stock solution (from Formulation Procedures—Part I, subpart A, above), and adding HCl (pH=3 to volume.
1. In a volumetric flask, transfer an aliquot ZnCl2 stock solution. Add enough HCl pH=3 (from step A.1) to volume and stir. Final concentration must be adjusted to every composition and peptide raw material (See F.1., below)
   Take a known weight of peptide and add the necessary weight (100% of total weight of excipient) of the immediately foregoing ZnCl2 solution to achieve the target concentration (1%, 2% or 10% (w/w)).
1. In a flask, transfer a weighed amount of (Aib^{8,35})HGLP-1(7-36)NH₂ (acetate salt). Add enough ZnCl2 solution of proper concentration (See F.1., below) to achieve the target composition (i.e.: 1%, 2% or 10% w/w) and stir to homogenize. The composition is then sterile filtered and sealed in a final container.
   Compositions with 1-2-10% peptide, ZnCl2 and upward adjusted pH (from a freeze-dried vial). Preparation of a ZnCl2 solution with the appropriate concentration:
1. In a volumetric flask, transfer an aliquot ZnCl2 stock solution. Add enough HCl pH=3 (from step A.1) to volume and stir. Final concentration must be adjusted to every composition and peptide raw material (See F. 1., below)
   Take a known weight of peptide and add the necessary volume (90% of total volume of excipient) of the immediately foregoing ZnCl2 solution related to achieve an intermediate product.
   1% compositions: Take a vial with 20 mg (Aib^{8,35})HGLP-1(7-36)NH₂ (step C) and add 1.8 mL of ZnCl2 solution of proper concentration (See F.1., below)
   2% compositions: Take a vial with 20 mg (Aib^{8,35})HGLP-1(7-36)NH₂ (step C) and add 0.9 mL of ZnCl2 solution of proper concentration (See F.1., below)
   10% compositions: Take a vial with 50 mg (Aib^{8,35})HGLP-1(7-36)NH₂ (step D) and add 0.40 mL of ZnCl2 solution of proper concentration (See F.1., below) Shake until dissolution.
      Add the necessary volume (10% of total volume of excipient) of diluted NaOH solution (depending on Peptide & Acetate content in peptide raw material, and target peptide concentration in composition) to achieve the target concentration & pH.
   1% compositions: Add 0.2 mL of NaOH solution of proper concentration
   2% compositions: Add 0.1 mL of NaOH solution of proper concentration
   10% compositions: Add 0.05 mL of NaOH solution of proper concentration Shake until dissolution.
      Compositions with 1-2-10-25% peptide, ZnCl2 and increasing pH (for 1, 2 & 10% compositions use liquid formulation process (see F.2., below); for 25% formulation use semi-solid/gel formulation process (see F.3., below.)).
      Preparation of a ZnCl2 solution with the appropriate concentration: taking an aliquot of the stock solution from 2.A., above, and adding HCl pH 3 to volume.
1. In a volumetric flask, transfer an aliquot ZnCl2 stock solution. Add enough HCl pH=3 (from step A.1) to volume and stir. Final concentration must be adjusted to every composition and peptide raw material (See F.1., below)
   Take a known weight of peptide and to add the necessary weight (75% of weight of total excipient) of solution of ZnCl2 related in last point, to achieve an intermediate product.
1. In a flask (or a syringe like container for 25% formulation by semi-solid/gel process, below), transfer a weighed amount of (Aib^{8,35})HGLP-1 (7-36)NH₂C. Add enough ZnCl2 solution of proper concentration (See F.1., below). Stir to homogenize (if liquid formulation process) or push-pull to homogenize (if semi-solid/gel process).
   Add the necessary weight (25% of total weight of excipient) of diluted NaOH solution (depending on Peptide & Acetate content in API, and target peptide concentration in composition) to achieve the target concentration & pH.
   Stir to homogenize (if liquid formulation process) or push-pull to homogenize (if semi-solid/gel process).
   Necessary weight/volume of excipient.

The total weight of excipient (E) to be added in each composition is be calculated as follows:
E=Weight of excipient (mg)=(A×100/T)−(A/P)
wherein:

A=content of pure peptide (mg);

T=target concentration of the composition; e.g., 1, 2, 10 or 25 if target is 1%, 2%, 10% or 25%, respectively; and

P=content of pure peptide (mg peptide/100 mg formulation) of raw material.

With respect to the total volume of excipient, the assumption that 1 mL=1 g is applied (e.g., re: freeze-dried compositions).

Volumes/Weights (W) of solution of ZnCl2 that must be added to each composition (mg solution or mL solution),

Compositions in which pH is not adjusted with base, e.g., NaOH: W=100% E;

Compositions in which pH is not adjusted with base, e.g., NaOH, and liquid formulation process (below) or semi-solid/gel process (also below): W=75% E;

Compositions with increased pH and freeze-dried reconstitution: W=90% E.

Volumes/Weights (W) of solution of NaOH that must be added to each composition (mg solution or mL solution),

Compositions with base-adjusted pH and liquid formulation process or semi-solid/gel process. W=25% E; and

Compositions with increased pH & increased pH and freeze-dried reconstitution: W=10% E.

F.1. Appropriate concentrations of ZnCl2

The appropriate concentration of ZnCl2 to be used in each composition is be calculated as follows:
Concentration ZnCl2 (mg/mL) or (mg/g)=(136.29×A)/(W×3339.76×R)
wherein:
A; Content of pure peptide (mg).

T: Target concentration of the composition, being 1, 2, 10 or 25 if target is 1%, 2%, 10% or 25%.

R: Molar Ratio Peptide/Zn, being R=1.5, for 1, 2 & 10% compositions or R=4.0 for 25% compositions.

W: Weight (g) or Volume (mL) of solution of ZnCl2 that must be added to each composition (g solution or mL solution).

F.2. Liquid Formulation Process

As noted herein, certain formulations of the current invention can be and were produced using the following liquid formulation process. By was of illustration, examples C5, C6, C7, C8, C9, C10, C11, C12 and C13 all were prepared essentially according to the following procedure. In each of the foregoing examples NaOH was used to adjust the pH of the composition.

1) The raw Peptide is accurately weighed into a glass vial.

2) The total composition amount and liquid volume are calculated according to Peptide content in peptide raw material and in view of the desired final composition.

3) The total liquid volume to be used in compositions is split up between the zinc and the NaOH solutions.

4) Zinc salt concentration is adjusted so that the total zinc amount needed is incorporated with 80% total liquid volume in the composition.

5) The zinc solution (either ZnCl2 or ZnAc₂.2H₂O) is accurately weighed and transferred to peptide vial.

6) The composition is stirred until homogenization and peptide content is determined in the intermediate product as an “in process” control.

7) Once the target peptide content in the intermediate product is concluded, the remaining intermediate bulk product is accurately weighed and the amount of NaOH solution calculated (the remaining 20% volume is added as NaOH solution).

8) The NaOH solution is accurately weighed and transferred to the vial.

9) The composition is stirred until homogenization.

F.3. Semi-solid/Gel Formulation Process

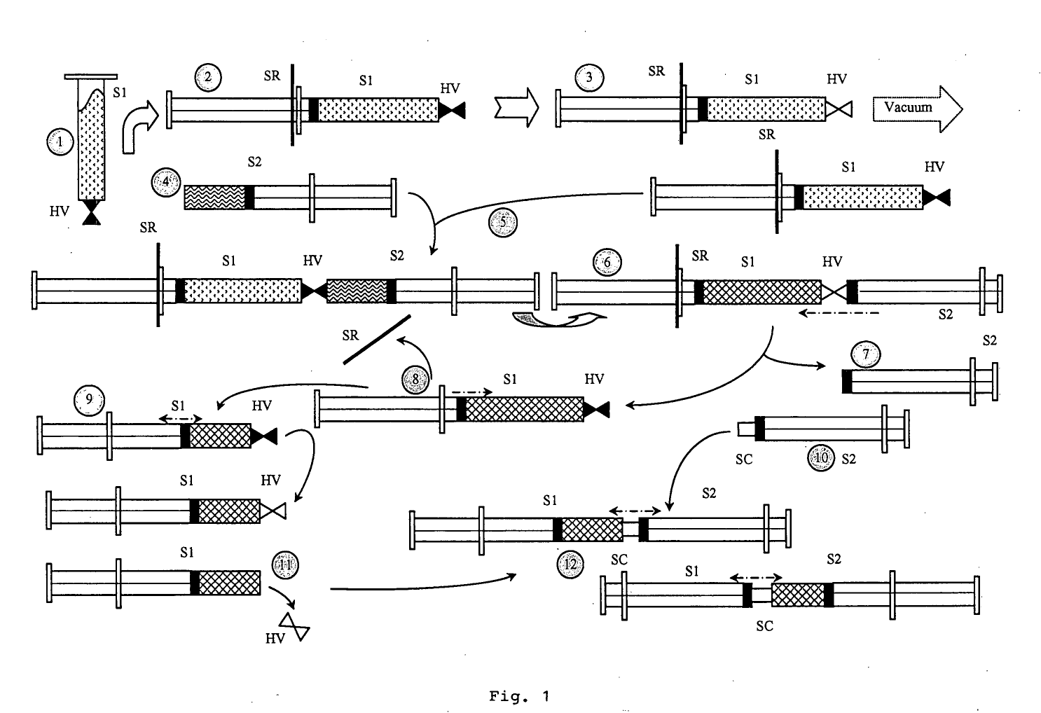

Reference is made to FIG. 1 herein, which depicts the steps followed in order to obtain homogeneous compositions for examples C1-C4.

a. Examples C1, and C2 were prepared essentially according to the following procedures.

1) The peptide is accurately weighed into the barrels of a disposable syringe S1. The syringe is previously fitted with a special two-way hand valve HV (I.D.=0.5 mm), the tubing placed inside the syringe Luer hole.

2) The syringe plunger is secured with a stainless steel rod SR.

3) HV in S1 is connected to a vacuum source and HV is opened. After 10 min HV is closed.

4) The solvent (Zinc solution) is accurately weighed into a second disposable syringe S2.

5) S2 is then connected to the free part of HV.

6) HV is opened and the solvent is pulled by the vacuum inside the powder container S1. 7) HV is closed and the solvent syringe S2 is removed, while the solvent hydrates the powder in S1.

8) SR is removed and the syringe plunger is slowly released (a fast movement would compress the mixture).

9) The syringe plunger is moved (push and pull), without opening HV, so that the powder mass is fully soaked by solvent.

10) A special two-way stainless connector SC (I.D.=1.0 mm) is placed in syringe S2 (the tubing placed inside the syringe Luer hole) and its plunger is pushed to the end.

11) HV in S1 is opened to vent vacuum and then HV is removed. The syringe plunger is moved so that air chamber in the syringe barrel is minimized.

12) S1 and S2 are connected by SC and the composition is kneaded from S1 to S2 through SC.

b) Compositions Including Peptide and Zinc Salt and NaOH Solutions

Examples C3 and C4 included NaOH in their compositions. The total liquid volume to be used in those batches is split up between the zinc and the NaOH solutions. Therefore, zinc salt concentration is adjusted so that the total zinc amount needed is incorporated with 50% total liquid volume in composition (step 4). The remaining 50% volume is added as NaOH solution and additional steps are required, as follows:

13) After homogenization, peptide content is determined in the intermediate product (from step 12) as an “in process” control.

14) Once the target peptide content in the intermediate product is concluded, the remaining intermediate bulk product is accurately weighed and the amount of NaOH solution calculated.

15) The NaOH solution is accurately weighed into a third disposable syringe S3.

16) The syringe plungers are pushed so that air chambers in the syringes are minimized. Both syringes are connected by SC and the composition is kneaded through SC.

Formulation Procedures—Part II

| Ex. | | | **Peptide:Zn | Peptide | |
|---|---|---|---|---|---|
| No. | *Peptide % | Solution | Ratio | Dose | |
| C1 | 10 | ZnCl₂ 0.846 mg/ml | 5.4:1 | 1 | mg |
| C2 | 5 | 0.40 mg ZnCl₂/mL | 5.4:1 | 1 | |
| C3 | 10 | 50% ZnCl₂ 1.69 mg/mL, 50% NaOH 1 mg/mL | 5.4:1 | 1 | |
| C4 | 10 | 50% ZnCl₂ 2.28 mg/mL, 50% NaOH 1 mg/mL | 4:1 | 1 | |
| C5 | 5 | 80% ZnCl₂ 0.674 mg/mL, 20% NaOH 3.81 mg/mL | 4:1 | 1 | |
| C6 | 2 | 80% ZnCl₂ 0.26 mg/mL, 20% NaOH 2.15 mg/mL | 5.4:1 | 1 | |
| C7 | 10 | 80% ZnCl₂ 3.81 mg/mL, 20% NaOH 4.47 mg/mL | 1.5:1 | 1 | |
| C8 | 10 | 80% ZnAc₂.2H₂O 2.3 mg/mL, 20% NaOH 6.1 mg/mL | 4:1 | 1 | |
| C9 | 2 | 80% ZnCl₂ 0.695 mg/mL, 20% NaOH 1.75 mg/mL | 1.5:1 | 1 | |
| C10 | 2 | 80% ZnAc₂.2H₂O 1.12 mg/mL, 20% NaOH 1.44 mg/mL | 1.5:1 | 1 | |
| C11 | 2 | 80% ZnCl₂ 0.695 mg/mL, 20% NaOH 1.75 mg/mL | 1.5:1 | 1 | |
| C12 | 1 | 80% ZnCl₂ 0.384 mg/mL, 20% NaOH 0.875 mg/mL | 1.5:1 | 1 | |
| C13 | 10 | 80% ZnCl₂ 3.85 mg/mL, 20% NaOH 4.47 mg/mL | 1.5:1 | 15 | |

*Target value shown. Actual value within 5% of target in all cases.
**Target value shown. Actual values within 10% of target in all cases

Determination of GLP-1 Receptor Affinity

A compound useful to practice the present invention can be tested for its ability to bind to the GLP-1 receptor using the following procedure.

Cell Culture:

RIN 5F rat insulinoma cells (ATCC-# CRL-2058, American Type Culture Collection, Manassas, Va.), expressing the GLP-1 receptor, were cultured in Dulbecco's modified Eagle's medium (DMEM) containing 10% fetal calf serum, and maintained at about 37° C. in a humidifed atmosphere of 5% CO₂/95% air.

Radioligand Binding:

Membranes were prepared for radioligand binding studies by homogenization of the RIN cells in 20 ml of ice-cold 50 mM Tris-HCl with a Brinkman Polytron (Westbury, N.Y.) (setting 6,15 sec). The homogenates were washed twice by centrifugation (39,000 g/10 min), and the final pellets were resuspended in 50 mM Tris-HCl, containing 2.5 mM MgCl₂, 0.1 mg/ml bacitracin (Sigma Chemical, St. Louis, Mo.), and 0.1% BSA. For assay, aliquots (0.4 ml) were incubated with 0.05 nM (¹²⁵I)GLP-1(7-36) (˜2200 Ci/mmol, New England Nuclear, Boston, Mass.), with and without 0.05 ml of unlabeled competing test peptides. After a 100 min incubation (25° C.), the bound (¹²⁵I)GLP-1(7-36) was separated from the free by rapid filtration through GF/C filters (Brandel, Gaithersburg, Md.), which had been previously soaked in 0.5% polyethyleneimine. The filters were then washed three times with 5 ml aliquots of ice-cold 50 mM Tris-HCl, and the bound radioactivity trapped on the filters was counted by gamma spectrometry (Wallac LKB, Gaithersburg, Md.). Specific binding was defined as the total (¹²⁵I)GLP-1(7-36) bound minus that bound in the presence of 1000 nM GLP1(7-36) (Bachem, Torrence, Calif.).

B. Determination of Solubility vs pH

B.1. Determination of Compound Solubility vs pH in Buffered Saline

A compound that may advantageously be used to practice the invention can be tested to determine its solubility in PBS at different pHs and temperatures using the following procedure.

A stock PBS buffered solution is made by dissolving one packet of pre-mixed powder (SIGMA, Product No.: P-3813) in one liter of de-ionized water to yield 10 mM phosphate-buffered saline with 138 mM NaCl, 2.7 mM KCl, and a pH of 7.4. PBS buffers with different pH values may be made by adjusting the pH of this stock solution with phosphoric acid and/or sodium hydroxide.

2 mg samples of a compound to be tested, e.g., a compound of Example 1, may be weighed into glass vials. Into each vial is added a 50 μl aliquot of PBS buffer at a certain pH. The solution is vortexed, and if necessary sonicated, until clear. For each pH tested the total volume of buffer needed to dissolve 2 mg of the compound is recorded and the solubility was calculated.

Peptide solutions that are clear at room temperature (20-25° C.) are placed in a refrigerator (4° C.) overnight and the solubility of the peptide at 4° C. is then examined.

B.2. Determination of Compound Solubility vs pH in Saline

A compound that may advantageously be used to practice the invention can be tested to determine its solubility in saline at different pH values and temperatures using the following procedure.

A stock saline solution is prepared by dissolving 9 grams of NaCl in one liter of de-ionized water. Saline solutions with different pH values are made by adjusting the pH of this stock solution with HCl and/or NaOH. 2 mg samples of a compound to be tested, e.g., a compound of example 1, are weighed into glass vials. Into each vial is added a 50 μl aliquot of saline solution at a certain pH. The vial is vortexed and, if necessary, sonicated until clear. For each tested pH the total volume of saline needed to dissolve 2 mg of the compound is recorded and the solubility is calculated.

Solutions that are clear at room temperature (20-25° C.) are placed in a refrigerator (4° C.) overnight and the solubility at 4° C. then examined.

B.3. Determination of Compound Solubility in Saline at pH 7.0

Compounds that may advantageously be used to practice the invention can be tested to determine their solubility at room temperature in saline having pH=7 using the following procedure.

Saline solution is prepared by dissolving 9 grams of NaCl in one liter of de-ionized water. A 2 mg sample of a compound to be tested, e.g., a compound of example 1, is weighed into a glass vial and 1 mL aliquots of saline are added, with vortexing and sonication, until clear. The total volume of saline used to dissolve 2 mg of peptide is recorded and the solubility at room temperature is calculated.

B.4. Determination of Compound Solubility in Saline at various pH

Compounds that may advantageously be used to practice the invention can be tested to determine their solubility at room temperature in saline solutions having various pH values using the following procedure.

A stock saline solution is prepared by dissolving 9 grams of NaCl in one liter of de-ionized water. Saline solutions having various pH values are obtained by treating aliquots of this stock saline solution with HCl and NaOH.

A 2 mg sample of a compound to be tested, e.g., the compound of example 1, is are weighed into glass vials. Aliquots of 50 μl of a saline buffer at a certain pH are added. The solution is vortexed and sonicated until clear. The total volume of buffer used to dissolve 2 mg of peptide is recorded and the solubility is calculated.

C. Determination of Aqueous Solubility of Compound vs Zinc Concentration

A compound that may advantageously be used to practice the invention can be tested to determine its solubility in pH 7 water at different zinc concentrations using the following procedure.

A stock zinc solution is prepared by dissolving ZnCl₂ in de-ionized water to a concentration of 100 mg/ml and adjusting the pH to 2.7 using HCl. Solutions having various ZnCl₂ concentrations (“Zn Test Solutions”) are prepared by making appropriate dilutions of the stock solution.

1 mg of a compound to be tested, e.g., the compound of example 1, is dissolved in 250 μl of each Zh Test Solution to yield a solution having 4 mg/ml of the compound. The pH of this solution is then adjusted using 0.2 N NaOH until white precipitates are observed to form. The precipitation solution was centrifuged and the mother liquor analyzed using HPLC. The UV absorption area of test compound peak is measured and the concentration of the test compound in the mother liquor is determined via comparison to a calibration curve.

As a representative example of a compound that may be used to practice the invention, the compound of Example 1 was tested in the immediately foregoing assay and the following results were obtained (aqueous saline, pH 7.0, room temperature):

TABLE 2

| ZnCl₂ concentration | Solubility |
|---|---|
| (μg/mL) | (mg/mL) |
| 0 | 5.788 |
| 80 | 0.0770 |
| 500 | 0.0579 |
| 1000 | 0.0487 |
| 1500 | 0.0668 |
| 2500 | 0.1131 |

F. Determination of pl Using IEF Gels

Invitrogen's Novex IEF pH3-10 gels may be used to measure the pl of GLP-1 peptides. Peptidyl compounds to be tested are dissolved in water to a concentration of 0.5 mg/ml. For each such compound, 5 μl of the resulting solution is mixed with 5 μl of Novex® Sample Buffer 2× (comprised of 20 mM Arginine free base, 20 mM Lysine free base and 15% Glycerol) and the resulting 10 μl sample solution is loaded onto the gel along with a protein standard sample.

Running buffers are also obtained from Invitrogen and the gel is run according to manufacture's instructions, generally as follows: 100 V constant for 1 hour, followed by 200 V constant for 1 hour, followed by 500 V constant for 30 minutes.

The gel is then fixed in 12% TCA containing 3.5% sulfosalicylic acid for 30 minutes, and then stained for 2 hours with Colloidal Coomassie Blue according to the instructions found on the Novexe Colloidal Blue Kit thereafter, then de-stained in water overnight.

The gel is scanned and analyzed by the program Fragment Analysis 1.2. pl's of unknown peptides are calculated relative to the pl's of standard compounds having pl values of: 10.7, 9.5, 8.3, 8.0, 7.8, 7.4, 6.9, 6.0, 5.3, 5.2, 4.5, 4.2, and 3.5.

G. In Vivo Assays

Compositions of the present invention can be tested to determine their ability to promote and enhanced effect in vivo using the following assays.

G.1. Experimental Procedure:

The day prior to the experiment, adult male Sprague-Dawley rats (Taconic, Germantown, N.Y.) that weighed approximately 300-350 g were implanted with a right atrial jugular cannula under chlorohydrate anesthetic. The rats were then fasted for 18 hours prior to the injection of the appropriate test composition or vehicle control at time 0. The rats continued to be fasted throughout the entire experiment.

A 0.5 mg/ml ZnCl₂ solution was prepared by dilution of a solution of 100 mg/ml ZnCl₂ in an HCl solution having pH 2.7 water. 1 mg of the compound of formula (I) ((Aib^{8,35})hGLP1(7-36)NH₂) was dissolved in 250 μl of this solution to yield a clear solution having 4 mg/ml of the compound and 0.5 mg/ml Zn at pH 4.

At time zero the rats were injected subcutaneously (sc) either with (a) the immediately forgoing solution of (Aib⁸,³⁵)hGLP-1(7-36)NH₂), or with vehicle control. In both cases the injection volume was very small (4-6 μL) and the dose of GLP-1 compound administered to the subject was 75 μg/kg. At the appropriate time after the sc injections a 500 μl blood sample was withdrawn via the intravenous (iv) cannula and the rats were given an iv glucose challenge to test for the presence of enhanced insulin secretion. The times of the glucose challenge were 0.25, 1, 6, 12 and 24 hours post-compound injection. After the initial blood sample was withdrawn glucose (1 g/kg) was injected iv and flushed in with 500 μl heparinized saline (10U/mL). Thereafter, 500 μl blood samples were withdrawn at 2.5, 5, 10 and 20 minutes post-glucose injection. Each of these was immediately followed by an iv injection of 500 μl heparinized saline (10U/mL) through the cannula. The blood samples were centrifuged, plasma was collected from each sample and the samples were stored at −20° C. until they were assayed for insulin content. The amount of insulin in each sample was determined using a rat insulin enzyme-linked immunosorbant assay (ELISA) kit (American Laboratory Products Co., Windham, N.H.).

Results:

A sustained insulin-enhancing activity was observed that was inducible by glucose injection over the full 24 hours of the experiment.

H. In Vivo Assays

There are a number of in vivo assays known in the art which enable the skilled artisan to determine a composition's ability to promote extended release of active compound in vivo.

H.1. By way of example, an aqueous test formulation was prepared comprising 1% (w/w) of the compound of formula (I) (acetate salt) in a buffered solution of ZnCL2 (peptide:Zn ratio=1.5:1.0).

A total of 6 male Beagle dogs, ages 42- 78 months and 14-21 kg bodyweight were maintained with free access to water and once daily food (approx. 400 g of dry standard diet (SAFE 125). The dogs were fasted 18 hours before administration of test composition.

The test composition was administered by subcutaneous route in the interscapular area by. The volume of administration (approx. 20 microliters per animal) was made by 0.3 mL Terumo syringes with 0.33-12 mm (BS=30M2913). A theoretical dose of approximately 0.2 mg peptide was thus achieved.

Blood samples were taken periodically, at approx. time=0, 8, 15, 30, 45 min, and 1, 2, 4, 8, and 12 hours, and 1, 2, 3, 4, 5, and 6 days after administration. The blood was rapidly chilled after sampling until centrifugation, and the plasma decanted and rapidly frozen pending assay. Determination of peptide plasma concentration was made after off line solid pase extraction, followed by on-line phase extraction coupled to LC-MS/MS, and the data obtained managed by Analyst v1.2 software.

Results:

The composition demonstrated an extended release of the active peptide for at least 2 days.

H.2. Similarly, a semi-solid composition was prepared comprising the compound according to formula (I) (acetate salt) (10% w/w), in a solution comprising 50% ZnCL₂ (2.28 mg/ml and 50% NaOH (1 mg/ml), resulting in a molar ratio, peptide:Zn, of approximately 4.0:1. The composition continued to release the active compound for approximately seven days.

Further assays with various permutations of the disclosed formulation have likewise been subject to in vivo assay and have confirmed that compositions of the present invention provide a useful drug delivery platform for the compound of formula (I).

The peptides used in this invention advantageously may be provided in the form of pharmaceutically acceptable salts. Examples of such salts include, but are not limited to, those formed with organic acids (e.g., acetic, lactic, maleic, citric, malic, ascorbic, succinic, benzoic, methanesulfonic, toluenesulfonic, or pamoic acid), inorganic acids (e.g., hydrochloric acid, sulfuric acid, or phosphoric acid), and polymeric acids (e.g., tannic acid, carboxymethyl cellulose, polylactic, polyglycolic, or copolymers of polylactic-glycolic acids). A typical method of making a salt of a peptide of the present invention is well known in the art and can be accomplished by standard methods of salt exchange. Accordingly, the TFA salt of a peptide of the present invention (the TFA salt results from the purification of the peptide by using preparative HPLC, eluting with TFA containing buffer solutions) can be converted into another salt, such as an acetate salt by dissolving the peptide in a small amount of 0.25 N acetic acid aqueous solution. The resulting solution is applied to a semi-prep HPLC column (Zorbax, 300 SB, C-8). The column is eluted with (1) 0.1N ammonium acetate aqueous solution for 0.5 hrs., (2) 0.25N acetic acid aqueous solution for 0.5 hrs. and (3) a linear gradient (20% to 100% of solution B over 30 min.) at a flow rate of 4 ml/min (solution A is 0.25N acetic acid aqueous solution; solution B is 0.25N acetic acid in acetonitrile/water, 80:20). The fractions containing the peptide are collected and lyophilized to dryness.

As is well known to those skilled in the art, the known and potential uses of GLP-1 are varied and multitudinous (See, Todd, J. F., et al., Clinical Science, 1998, 95, pp. 325-329; and Todd, J. F. et al., European Journal of Clinical Investigation, 1997, 27, pp. 533-536). Thus, the administration of the compounds of this invention for purposes of eliciting an agonist effect can have the same effects and uses as GLP-1 itself. These varied uses of GLP-1 may be summarized as follows, treatment of: Type I diabetes, Type II diabetes, obesity, glucagonomas, secretory disorders of the airway, metabolic disorder, arthritis, osteoporosis, central nervous system diseases, restenosis, neurodegenerative diseases, renal failure, congestive heart failure, nephrotic syndrome, cirrhosis, pulmonary edema, hypertension, disorders wherein the reduction of food intake is desired, as well as the various other conditions and disorders discussed herein. Accordingly, the present invention includes within its scope pharmaceutical compositions as defined herein comprising, as an active ingredient, a compound of formula (I).

The dosage of active ingredient in the formulations of this invention may be varied; however, it is necessary that the amount of the active ingredient be such that a suitable dosage is obtained. The selected dosage depends upon the desired therapeutic effect, on the route of administration, and on the duration of the treatment, and normally will be determined by the attending physician. In general, an effective dosage for the activities of this invention is in the range of 1×10⁻⁷ to 200 mg/kg/day, preferably 1×10⁻⁴ to 100 mg/kg/day, which can be administered as a single dose or divided into multiple doses.

The formulations of this invention are preferably administered parenterally, e.g., intramuscularly, intraperitoneally, intravenously, subcutaneously, and the like.

Preparations according to this invention for parenteral administration include sterile aqueous or non-aqueous solutions, suspensions, gels, or emulsions, provided that the desired in vivo release profile is achieved. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use.

Unless defined otherwise, all technical and scientific terms used herein have the same meaning as commonly understood by one of ordinary skill in the art to which this invention belongs. Also, all publications, patent applications, patents and other references mentioned herein are incorporated by reference.

What is claimed is:

1. A pharmaceutical composition comprising an analog according to the formula:
[Aib^{8,35}]hGLP-1(7-36)NH₂;
together with zinc and a pharmaceutically acceptable carrier or diluent.

2. A pharmaceutical composition according to claim 1, wherein said zinc is present in a concentration from 0.0005 mg/mL to 50 mg/mL.

3. A pharmaceutical composition according to claim 2, wherein said zinc is present in a concentration from 0.01 mg/mL to 0.50 mg/mL.

4. A pharmaceutical composition according to claim 1, wherein said diluent comprises a pharmaceutically acceptable aqueous solution.

5. A pharmaceutical composition according to claim 4, wherein said diluent comprises sterile water.

6. A pharmaceutical composition according to claim 1, wherein said pharmaceutical composition comprises an aqueous mixture, suspension or solution, and wherein said compound of formula (I) is present at a concentration of approximately 0.5%-30% (w/w).

7. A pharmaceutical composition according to claim 6, wherein the concentration of said compound of formula (I) in said aqueous mixture, suspension or solution is approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (w/w).

8. A pharmaceutical composition according to claim 7, wherein the concentration of said compound of formula (I) in said aqueous solution is approximately 1%, 2%, 3%, 4%, 5%, 6%, 7%, 8%, 9%, 10%, 11%, 14%, 15%, 16%, 19%, 20%, 21%, 22%, 23%, 24%, 25%, 26%, 29%, or 30% (w/w).

9. A pharmaceutical composition according to claim 8, wherein the concentration of said compound of formula (I) in said aqueous solution is approximately 1%, 2%, 3%, 4%, 5%, 6%, 9%, 10%, 11%, 22%, 23%, 24%, 25%, or 26% (w/w).

10. A pharmaceutical composition according to claim 9, wherein the concentration of said compound of formula (I) in said aqueous solution is approximately 1%, 2%, 3%, 4%, 5%, 6%, 10%, 22%, 23%, 24%, 25%, or 26% (w/w).

11. A pharmaceutical composition according to claim 10, wherein the concentration of said compound of formula (I) in said aqueous solution is approximately 1%, 2%, 5%, 10%, 23% or 25% (w/w).

12. A pharmaceutical composition according to claim 6, wherein the molar ratio of said compound of formula (I) to zinc in said pharmaceutical composition ranges from approximately 6:1 to approximately 1:1.

13. A pharmaceutical composition according to claim 12, wherein said ratio ranges from approximately 5.5:1 to approximately 1:1.

14. A pharmaceutical composition according to claim 13, wherein said ratio ranges from approximately 5.4:1 to approximately 1.5:1.

15. A pharmaceutical composition according to claim 14, wherein said ratio is approximately 5.4:1, 4.0:1, or 1.5:1.

16. A pharmaceutical composition according to claim 15, wherein said ratio is approximately 1.5:1.

17. A pharmaceutical composition according to claim 6, wherein said zinc is provided as zinc chloride or zinc acetate.

18. A pharmaceutical composition according to claim 6, wherein said zinc acetate is provided as ZnAc₂.2 H₂O.

19. A pharmaceutical composition according to claim 1, wherein pH of said pharmaceutical composition is adjusted using a base.

20. A pharmaceutical composition according to claim 19, said pH adjustment is made using NaOH.

21. A pharmaceutical composition according to claim 20, wherein the pH of said pharmaceutical composition is adjusted with NaOH such that, when diluted to approximately ½ initial concentration using 0.9% NaCl, a pH value of approximately 5.0-5.5 is obtained.

22. A pharmaceutical composition according to claim 6, wherein the pH of said pharmaceutical composition is adjusted using a base.

23. A pharmaceutical composition according to claim 22, wherein said pH adjustment is made using NaOH.

24. A pharmaceutical composition according to claim 23, wherein the pH of said pharmaceutical composition is adjusted with NaOH such that, when diluted to approximately ½ initial concentration using 0.9% NaCl, a pH value of approximately 5.0-5.5 is obtained.

25. A pharmaceutical composition according to claim 1, wherein the compound according to formula (I) is released within a subject in need thereof for an extended period of time.

26. A pharmaceutical composition according to claim 25, wherein said release of said compound extends for at least from approximately one hour to approximately 12 hours

27. A pharmaceutical composition according to claim 26, said release of said compound extends for at least approximately 24 hours.

28. A pharmaceutical composition according to claim 27, wherein the compound according to formula (I) is released for at least approximately 48 hours, more preferably at least approximately 72 hours, more preferably still at least approximately 96 hours.

29. A pharmaceutical composition according to claim 28, wherein the compound according to formula (I) is released within a subject for at least approximately 5 to approximately 7 days, more preferably at least approximately 14 days, more preferably at least approximately 2 weeks, more preferably still at least approximately 4 weeks.

30. A pharmaceutical composition according to claim 6, wherein the compound according to formula (I) is released within a subject in need thereof for an extended period of time.

31. A pharmaceutical composition according to claim 30, wherein said release of said compound extends for at least from approximately one hour to approximately 12 hours

32. A pharmaceutical composition according to claim 31, said release of said compound extends for at least approximately 24 hours.

33. A pharmaceutical composition according to claim 32, wherein the compound according to formula (I) is released for at least approximately 48 hours, more preferably at least approximately 72 hours, more preferably still at least approximately 96 hours.

34. A pharmaceutical composition according to claim 33, wherein the compound according to formula (I) is released within a subject for at least approximately 5 to approximately 7 days, more preferably at least approximately 14 days, more preferably at least approximately 2 weeks, more preferably still at least approximately 4 weeks.

35. A pharmaceutical composition according to any one of claims 25-29, wherein said subject is a mammal, preferably a human,

36. A pharmaceutical composition according to any one of claims 30-34, wherein said subject is a mammal, preferably a human.

37. A method of eliciting a GLP-1 agonist effect, said method comprising contacting a receptor of the GLP-1(7-36)NH2 ligand with the compound according to formula (I), said compound according to formula (I) being provided to said receptor, directly or indirectly, via a composition according to claim 1.

38. A method of eliciting an agonist effect from a GLP-1 receptor in a subject in need thereof, said method comprising administering to said subject a pharmaceutical composition according to claim 1.

39. The method according to claim 38, wherein said receptor of the GLP-1(7-36)NH₂ ligand is present in an animal subject.

40. The method according to claim 39, wherein said subject is a human being.

41. The method according to claim 40, wherein said human subject is afflicted with, or at risk of developing, a disease or condition selected from the group consisting of Type I diabetes, Type II diabetes, gestational diabetes, obesity, excessive appetite, insufficient satiety, and metabolic disorder.

42. The method according to claim 41, wherein said said disease is Type I diabetes or Type II diabetes.

43. The method according to claim 40, wherein said human subject is afflicted with, or at risk of developing, a disease or condition selected from the group consisting of glucagonomas, secretory disorders of the airway, arthritis, osteoporosis, central nervous system disease, restenosis, neurodegenerative disease, renal failure, congestive heart failure, nephrotic syndrome, cirrhosis, pulmonary edema, hypertension, and disorders wherein the reduction of food intake is desired, a disease or disorder of the central nervous system, Parkinson's Disease, Alzheimer's Disease, Huntington's Disease, ALS, stroke, ADD, and neuropsychiatric syndromes, irritable bowel syndrome, myocardial infarction, stroke, acute coronary syndrome, post-surgical catabolic changes, hibernating myocardium or diabetic cardiomyopathy, insufficient urinary sodium excretion, excessive urinary potassium concentration, conditions or disorders associated with toxic hypervolemia, (e.g., renal failure, congestive heart failure, nephrotic syndrome, cirrhosis, pulmonary edema, and hypertension), polycystic ovary syndrome, respiratory distress, nephropathy, left ventricular systolic dysfunction, gastrointestinal disorders such as diarrhea, postoperative dumping syndrome and irritable bowel syndrome, critical illness polyneuropathy (CIPN), systemic inflammatory response syndrome (SIRS), dyslipidemia, organ tissue injury caused by reperfusion of blood flow following ischemia, and coronary heart disease risk factor (CHDRF) syndrome.

44. A method of converting liver stem/progenitor cells into functional pancreatic cells, of preventing beta-cell deterioration and of stimulating beta-cell proliferation, of suppressing plasma blood levels of norepinepherine, of inducing an inotropic response and of increasing cardiac contractility, of improving nutrition via a non-alimentary route, of pre-treating a subject to undergo an endoscopic procedures, and of modulating triglyceride levels, in a subject in need thereof, said method comprising administering to said subject a pharmaceutical composition according to claim 1.

45. The method according to claim 44, wherein said subject is a mammalian animal, more preferably a primate, more preferably still a human being.

46. A method of eliciting a GLP-1 agonist effect, said method comprising contacting a receptor of the GLP-1(7-36)NH2 ligand with the compound according to formula (I), said compound according to formula (I) being provided to said receptor, directly or indirectly, via a composition according to claim 6.

47. A method of eliciting an agonist effect from a GLP-1 receptor in a subject in need thereof, said method comprising administering to said subject a pharmaceutical composition according to claim 6.

48. The method according to claim 47, wherein said receptor of the GLP-1(7-36)NH₂ ligand is present in an animal subject.

49. The method according to claim 48, wherein said subject is a human being.

50. The method according to claim 49, wherein said human subject is afflicted with, or at risk of developing, a disease or condition selected from the group consisting of Type I diabetes, Type II diabetes, gestational diabetes, obesity, excessive appetite, insufficient satiety, and metabolic disorder.

51. The method according to claim 50, wherein said disease is Type I diabetes or Type II diabetes.

52. The method according to claim 49, wherein said human subject is afflicted with, or at risk of developing, a disease or condition selected from the group consisting of glucagonomas, secretory disorders of the airway, arthritis, osteoporosis, central nervous system disease, restenosis, neurodegenerative disease, renal failure, congestive heart failure, nephrotic syndrome, cirrhosis, pulmonary edema, hypertension, and disorders wherein the reduction of food intake is desired, a disease or disorder of the central nervous system, Parkinson's Disease, Alzheimer's Disease, Huntington's Disease, ALS, stroke, ADD, and neuropsychiatric syndromes, irritable bowel syndrome, myocardial infarction, stroke, acute coronary syndrome, post-surgical catabolic changes, hibernating myocardium or diabetic cardiomyopathy, insufficient urinary sodium excretion, excessive urinary potassium concentration, conditions or disorders associated with toxic hypervolemia, (e.g., renal failure, congestive heart failure, nephrotic syndrome, cirrhosis, pulmonary edema, and hypertension), polycystic ovary syndrome, respiratory distress, nephropathy, left ventricular systolic dysfunction, gastrointestinal disorders such as diarrhea, postoperative dumping syndrome and irritable bowel syndrome, critical illness polyneuropathy (CIPN), systemic inflammatory response syndrome (SIRS), dyslipidemia, organ tissue injury caused by reperfusion of blood flow following ischemia, and coronary heart disease risk factor (CHDRF) syndrome.

53. A method of converting liver stem/progenitor cells into functional pancreatic cells, of preventing beta-cell deterioration and of stimulating beta-cell proliferation, of suppressing plasma blood levels of norepinepherine, of inducing an inotropic response and of increasing cardiac contractility, of improving nutrition via a non-alimentary route, of pre-treating a subject to undergo an endoscopic procedures, and of modulating triglyceride levels, in a subject in need thereof, said method comprising administering to said subject a pharmaceutical composition according to claim 6.

54. The method according to claim 53, wherein said subject is a mammalian animal, more preferably a primate, more preferably still a human being.
